(19) United States
(12) Patent Application Publication
(10) Pub. No.: US 2025/0090847 A1
(43) Pub. Date: Mar. 20, 2025

(54) DEVICE FOR INHIBITING TUMOR PROLIFERATION BY USING ELECTRIC FIELD, AND CONTROL METHOD AND APPARATUS THEREFOR

(71) Applicant: ANTITUMOR AND HEALTH RECONSTRUCTION BIOTECHNOLOGY CO., LTD, Changsha (CN)

(72) Inventors: Jian LU, Changsha (CN); Lingzhi XIONG, Changsha (CN)

(21) Appl. No.: 18/568,295

(22) PCT Filed: Feb. 10, 2022
(86) PCT No.: PCT/CN2022/075854

§ 371 Date: Aug. 7, 2024

(30) Foreign Application Priority Data
Jun. 8, 2021 (CN) 202110636355.X

(51) Int. Cl. A61N 1/36

(52) U.S. Cl. CPC A61N 1/36031; A61N 1/36034

(57) ABSTRACT

Provided in the present application are a device for inhibiting tumor proliferation by using an electric field, and a control method and apparatus therefor. The device for inhibiting tumor proliferation by using an electric field comprises an alternating-current voltage source and an electrode patch pair, wherein two electrode patches in the electrode patch pair are used for being attached to the skin of a user, and the alternating-current voltage source is connected to the electrode patch pair to apply an alternating-current voltage to the human tissue between the two electrode patches. The method comprises: acquiring a measured temperature value of a contact area between an electrode patch and skin, and determining, according to the measured temperature value, a target temperature value and a target temperature interval, a set output voltage value provided for an alternating-current voltage source, wherein the target temperature value is within the target temperature interval.

### CROSS REFERENCES TO RELATED APPLICATIONS

This application claims priority to Chinese Patent Application No. 202110636355.X, filed on Jun. 8, 2021 and entitled “DEVICE FOR INHIBITING TUMOR PROLIFERATION BY USING ELECTRIC FIELD, AND CONTROL METHOD AND APPARATUS THEREFOR”, which is incorporated by reference herein in its entirety.

### TECHNICAL FIELD

The present disclosure relates to the field of automatic control, and specifically relates to a method and apparatus for controlling a device for inhibiting tumor proliferation by using an electric field, and a device for inhibiting tumor proliferation by using an electric field.

### BACKGROUND

This section is intended to provide background or context to embodiments of the disclosure as set forth in the claims. What is described herein is not admitted to be prior art by virtue of its inclusion in this section.

A device for inhibiting tumor proliferation by using an electric field works by applying an alternating current (AC) electric field to a lesion region. Specifically, at least one pair of electrode patches are attached to a user's skin surface, and then an AC voltage is applied to the lesion region through the electrode patches from an AC voltage source.

Cells in the late anaphase or telophase stages of cell division are vulnerable to damage by AC electric fields that have specific frequencies and field strength characteristics. Therefore, selective destruction of rapidly dividing cells can be accomplished by imposing an AC electric field in a target region for extended periods of time. Some of the cells that divide while the field is applied will be damaged, but the cells that do not divide will not be harmed. This selectively damages rapidly dividing cells like tumor cells, but does not harm normal cells that are not dividing.

### SUMMARY

Embodiments of the disclosure provide a method and apparatus for controlling a device for inhibiting tumor proliferation by using an electric field, and a device for inhibiting tumor proliferation by using an electric field. Based on the control method and apparatus, and the device for inhibiting tumor proliferation by using an electric field, a user could receive a prolonged high-field-strength treatment.

Embodiments of the disclosure provide a method of controlling a device for inhibiting tumor proliferation by using an electric field, the device for inhibiting tumor proliferation by using an electric field including a pair of electrode patches configured to be attached to a skin of a user and an alternating current (AC) voltage source connected to the pair of electrode patches to apply an AC voltage to a human tissue of the user between the electrode patches, where the method includes: acquiring a measured temperature value of a contact region between the skin and at least one of the electrode patches; and determining an output voltage setting value for the AC voltage source according to the measured temperature value, a target temperature value, and a target temperature range, where the target temperature value is within the target temperature range.

Embodiments of the disclosure further provide an apparatus for controlling a device for inhibiting tumor proliferation by using an electric field, the device for inhibiting tumor proliferation by using an electric field including a pair of electrode patches configured to be attached to a skin of a user and an AC voltage source connected to the pair of electrode patches to apply an AC voltage to a human tissue of the user between the electrode patches, where the apparatus includes: an acquisition module configured to acquire a measured temperature value of a contact region between the skin and at least one of the electrode patches; and a second adjustment module configured to determine an output voltage setting value for the AC voltage source according to the measured temperature value, a target temperature value, and a target temperature range, where the target temperature value is within the target temperature range.

Embodiments of the disclosure further provide an apparatus for controlling a device for inhibiting tumor proliferation by using an electric field, the apparatus including at least one processor and a memory communicatively connected to the at least one processor, the memory having instructions executable by the at least one processor stored therein, where the instructions, when executed by the at least one processor, cause the at least one processor to implement the aforesaid control method.

Embodiments of the disclosure provide a device for inhibiting tumor proliferation by using an electric field, including a pair of electrode patches configured to be attached to a skin of a user and an AC voltage source connected to the pair of electrode patches to apply an AC voltage to a human tissue of the user between the electrode patches, where the device further includes the aforesaid control apparatus.

At least one of the aforesaid technical solutions adopted in embodiments of the disclosure can achieve the following beneficial effects. Without considering other factors, the greater the output voltage setting value provided to the AC voltage source, the greater the AC voltage applied by the two electrode patches to the human issue and the better the therapeutic effect of the device for inhibiting tumor proliferation by using an electric field. On the other hand, the higher the temperature of the contact region between the skin and the electrode patch, the higher the probability of alarm and shutdown of the device for inhibiting tumor proliferation by using an electric field. According to embodiments of the disclosure, during the operation of the device for inhibiting tumor proliferation by using an electric field, the output voltage setting value for the AC voltage source is dynamically regulated in real time according to the measured temperature value of the contact region between the skin and at least one of the electrode patches, the target temperature value, and the target temperature range, whereby the device for inhibiting tumor proliferation by using an electric field is enabled to output high voltage for a long time, thus enhancing user experiences and improving therapeutic effects. Furthermore, the relationship of the measured temperature value with the target temperature value and the target temperature range is required to be referred to for adjustment of the output voltage setting value, thus smoother fluctuation of the output voltage setting value can be achieved.

It should be understood that the above description is a summary of the technical solutions of the disclosure only for the purpose of facilitating a better understanding of the technical means of the disclosure so that the disclosure could be implemented according to the description in the specification. Specific embodiments of the disclosure are given below to render the above and other objects, features, and advantages of the disclosure more clear.

### BRIEF DESCRIPTION OF THE DRAWINGS

By reading the detailed description of exemplary embodiments below, a person of ordinary skill in the art would understand the advantages and benefits described herein, as well as other advantages and benefits. The accompanying drawings are only for the purpose of illustrating exemplary embodiments and are not intended to be any limitation of the disclosure. Moreover, throughout the accompanying drawings, the same reference numerals are designated for the same components.

FIG. 1 is a structural block diagram of a device for inhibiting tumor proliferation by using an electric field according to an embodiment of the disclosure;

FIG. 2 is a schematic diagram of a flowchart of a method of controlling a device for inhibiting tumor proliferation by using an electric field according to an embodiment of the disclosure;

FIG. 3 is a schematic structural diagram of an apparatus for controlling a device for inhibiting tumor proliferation by using an electric field according to an embodiment of the disclosure; and

FIG. 4 is a schematic structural diagram of an apparatus for controlling a device for inhibiting tumor proliferation by using an electric field according to another embodiment of the disclosure.

In the drawings, the same or corresponding reference numerals designate the same or corresponding parts.

### DETAILED DESCRIPTION

Exemplary embodiments of the present disclosure will be described below in more details with reference to the accompanying drawings. Although the accompanying drawings illustrate exemplary embodiments of the present disclosure, it should be understood that the present disclosure may be implemented in various ways, and should not be construed to be limited to the embodiments described herein. Rather, these embodiments are provided to facilitate more thorough understanding of the present disclosure so that the scope of the present disclosure could be fully conveyed to a person of ordinary skill in the art.

In the disclosure, it should be understood that terms such as “include” or “have” are intended to indicate the existence of the characters, digits, steps, actions, components, parts disclosed in the specification or any combination thereof, without excluding the existence of one or more other characters, digits, steps, actions, components, parts or any combination thereof.

Furthermore, it should be noted that, in case of no conflict, the embodiments of the disclosure and features therein may be combined with each other in any manner. The disclosure will be described in detail below with reference to the accompanying drawings and embodiments.

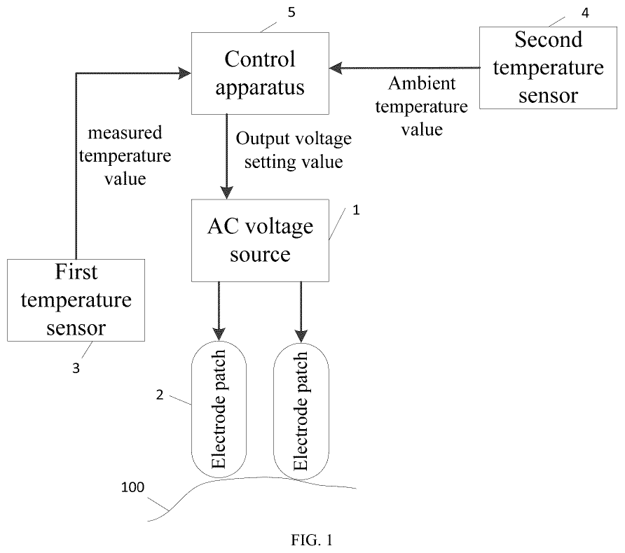

Referring to FIG. 1, a device for inhibiting tumor proliferation by using an electric field according to some embodiments of the disclosure includes an alternating current (AC) voltage source 1, a pair of electrode patches, a control apparatus 5, a first temperature sensor 3, and a second temperature sensor 4. The first temperature sensor 3 is configured to measure the temperature of a contact region between a skin 100 and at least one of electrode patches 2 of the device for inhibiting tumor proliferation by using an electric field to obtain a measured temperature value. The second temperature sensor 4 is configured to measure an ambient temperature to obtain an ambient temperature value. The two electrode patches 2 in the pair of electrode patches are respectively attached to different positions of the user skin 100. The AC voltage source 1 is connected to the pair of electrode patches to apply an AC voltage to a human tissue of the user between the two electrode patches 2. The control apparatus 5 is configured to provide an output voltage setting value to the AC voltage source 1 to set the output voltage of the AC voltage source 1.

It should be noted that although one pair of electrode patches 2 are shown in FIG. 1, the device for inhibiting tumor proliferation by using an electric field may include two or more pairs of electrode patches. The control method proposed in the disclosure may be applied to each pair of electrode patches. The multiple pairs of electrode patches may work simultaneously or alternately.

In some possible variations, the first temperature sensor 3 and/or the second temperature sensor 4 are components independent of the device for inhibiting tumor proliferation by using an electric field, which are configured to provide the measured temperature value and/or the ambient temperature value for the control apparatus 5 in a wired or wireless manner.

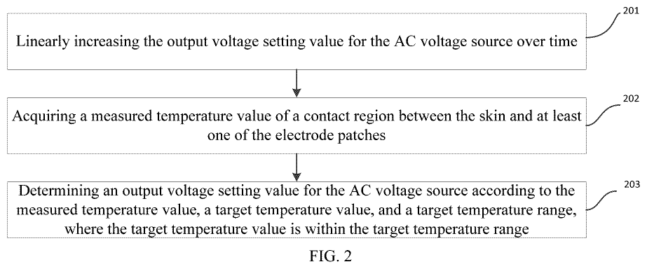

FIG. 2 is a schematic diagram of a flowchart of a method of controlling a device for inhibiting tumor proliferation by using an electric field according to an embodiment of the disclosure. The method is used to regulate an output voltage of an AC voltage source. In the flowchart, from a hardware perspective, the execution body may be a control apparatus in the device for inhibiting tumor proliferation by using an electric field; from a software perspective, the execution body may be correspondingly a program installed in a control apparatus of the device for inhibiting tumor proliferation by using an electric field. The control method includes the following steps 202 and 203.

Step 202: a measured temperature value of a contact region between the skin 100 and at least one of the electrode patches is acquired.

Step 203: an output voltage setting value for the AC voltage source is determined according to the measured temperature value, a target temperature value, and a target temperature range, where the target temperature value is within the target temperature range.

The target temperature value may be an upper limit of the target temperature range, a lower limit of the target temperature range, or a temperature setting value between the upper limit of the target temperature range and the lower limit of the target temperature range.

Without considering other factors, the greater the output voltage setting value provided to the AC voltage source, the greater the AC voltage applied by the two electrode patches to the human issue and the better the therapeutic effect of the device for inhibiting tumor proliferation by using an electric field. On the hand, the higher the temperature of the contact region between the skin 100 and the electrode patch, the higher the probability of alarm and shutdown of the device for inhibiting tumor proliferation by using an electric field.

According to embodiments of the disclosure, during the operation of the device for inhibiting tumor proliferation by using an electric field, the output voltage setting value for the AC voltage source is dynamically regulated in real time according to the measured temperature value of the contact region between the skin 100 and at least one of the electrode patches, the target temperature value, and the target temperature range. By comparing the measured temperature value to the target temperature value and the target temperature range and dynamically adjusting the output voltage setting value according to the comparison result, the measured temperature value can keep stable within the target temperature range for a long time, and the output voltage of the AC voltage source can thus also be maintained relatively high for a long time, whereby the device for inhibiting tumor proliferation by using an electric field is enabled to output high voltage for a long time, thus enhancing user experiences and improving therapeutic effects.

Furthermore, the relationship of the measured temperature value with the target temperature value and the target temperature range is required to be referred to for adjustment of the output voltage setting value, thus smoother fluctuation of the output voltage setting value can be achieved.

Generally, the higher the output voltage setting value, the higher the output voltage of the AC voltage source. It can be considered that an output voltage value of the AC voltage source is approximately equal to the output voltage setting value provided to the AC voltage source.

In some embodiments, determining the output voltage setting value for the AC voltage source according to the measured temperature value, the target temperature value, and the target temperature range includes: after the measured temperature value falls within the target temperature range, if the measured temperature value becomes lower than a lower limit of the target temperature range, updating the output voltage setting value according to an error value between the measured temperature value and the target temperature value and a differential value of the error value.

Generally, after the device for inhibiting tumor proliferation by using an electric field starts working, the control apparatus output the output voltage setting value that increases gradually, which in turn causes the temperatures of the electrode patches to gradually increase. After the measured temperature value falls within the target temperature range for the first time, under normal circumstances, the control apparatus should keep the measured temperature value within the target temperature range as much as possible. During the operation of the device for inhibiting tumor proliferation by using an electric field, the measured temperature value may sometimes exceed the target temperature range, but most of the time the measured temperature value can be kept within the target temperature range.

When unexpected situations such as a sudden drop in ambient temperature occur (such as turning off the heating in the room or opening the window of the room, causing the temperatures of the electrode patches to drop), a rapid decrease of the measured temperature value may be caused. A slight looseness of the electrode patch in contact with the skin 100 may also cause a decrease in the measured temperature value. The output voltage setting value can be rapidly increased according to the error value between the measured temperature value and the target temperature value, which in turn causes rapid increase of the temperatures of the electrode patches. A larger output voltage setting value can lead to a better therapeutic effect. However, it is desired to avoid voltage overshoot during the process of increasing the output voltage setting value, that is, to prevent the temperature from rising too fast and thus exceeding the maximum allowable temperature value. Therefore, differential adjustment is introduced in the process of increasing the output voltage setting value. The function of differential adjustment is to suppress the occurrence of voltage overshoot. Differential adjustment is beneficial in reducing the increasing speed of the output voltage setting value at the end of the process, thereby preventing overshoot.

For example, updating the output voltage setting value according to the error value between the measured temperature value and the target temperature value, and the differential value of the error value includes: updating the output voltage setting value according to the following formula:

$$u ( t ) = u ( t - 1 ) + K 5 * e ( t ) + K 6 * de ( t ) / dt ,$$

where u(t−1) is the output voltage setting value for the current adjustment period, u(t) is the output voltage setting value for the next adjustment period, e(t) is the error value between the measured temperature value and the target temperature value for the current adjustment period, de(t)/dt is the differential value of the error value for the current adjustment period, and K5 and K6 are positive proportional coefficients.

That is, a proportional-differential adjustment approach is used to control the rapid increase of the output voltage setting value and thus avoid overshoot. It could be understood that, the specific way of updating the output voltage setting value according to the error value between the measured temperature value and the target temperature value, and the differential value of the error value is not limited herein, as long as the output voltage setting value can be controlled to be increased rapidly without overshoot.

In some embodiments, determining the output voltage setting value for the AC voltage source according to the measured temperature value, the target temperature value, and the target temperature range includes: after the measured temperature value falls within the target temperature range, if the measured temperature value becomes greater than an upper limit of the target temperature range, updating the output voltage setting value according to an error value between the measured temperature value and the target temperature value, and an integral value of the error value, where an integration start point for the integral value is located in an adjustment period in which the measured temperature value becomes greater than the upper limit of the target temperature range.

After the measured temperature value falls within the target temperature range, under normal circumstances, the control apparatus should keep the measured temperature value within the target temperature range as much as possible based on a certain mechanism. When unexpected situations such as sudden increase in ambient temperature occur (such as turning on the heating in the room or closing the window of the room, causing the temperatures of the electrical patches to increase), a rapid increase in the measured temperature value may be caused. The output voltage setting value can be rapidly decreased according to the error value between the measured temperature value and the target temperature value, which in turn causes rapid decrease of the temperatures of the electrode patches, thereby preventing the electrode patches from being overheated to scald the user's skin 100 or trigger alarm and thus shutdown. In this sense, it is desired to reduce the voltage as soon as possible to prevent the overtemperature. Voltage downward adjustment can be speeded up by integral adjustment according to the error value, because the larger the temperature deviation, the larger the integral adjustment value. Moreover, the voltage downward adjustment mainly aims to suppress the increase in temperature, thus even if the temperature is moderately over-adjusted, there is no problem. Therefore, temperature overshoot caused by integral adjustment is acceptable.

For example, updating the output voltage setting value according to the error value between the measured temperature value and the target temperature value and the integral value of the error value includes: updating the output voltage setting value according to the following formula:

$$u ( t ) = u ( t - 1 ) + K 3 * e ( t ) + K 4 \int e ( t ) dt ,$$

where u(t−1) is the output voltage setting value for the current adjustment period, u(t) is the output voltage setting value for the next adjustment period, e(t) is the error value between the measured temperature value and the target temperature value in the current adjustment period, ∫e(t)dt is the integral value of the error value, and K3 and K4 are positive proportional coefficients.

That is, a proportional-integral adjustment approach is used to quickly reduce the temperatures of the electrode patches, where even if the temperature is moderately over-adjusted, it is acceptable. The specific way of updating the output voltage setting value according to the error value between the measured temperature value and the target temperature value and the integral value of the error value is not limited herein, as long as the temperature of the electrode patches can be rapidly reduced.

In some embodiments, determining an output voltage setting value for the AC voltage source according to the measured temperature value, the target temperature value, and the target temperature range includes: if the measured temperature value is within the target temperature range, maintaining the output voltage setting value unchanged.

The objective of increasing or decreasing the output voltage setting value as mentioned above is to cause the measured temperature value to be equal to the target temperature value. However, by keeping the output voltage setting value unchanged once the measured temperature value falls within the target temperature range, the fluctuation of the measured temperature value can be reduced and the control logic can be simplified.

In some embodiments, before determining the output voltage setting value for the AC voltage source according to the measured temperature value, the target temperature value, and the target temperature range, the method further includes step 201 of linearly increasing the output voltage setting value for the AC voltage source over time. Accordingly, determining the output voltage setting value between the pair of electrode patches according to the measured temperature value, the target temperature value, and the target temperature range includes: before the measured temperature value falls within the target temperature range for the first time, updating the output voltage setting value according to the error value between the measured temperature value and the target temperature value.

That is, the output voltage setting value is increased linearly over time in the early stage of initiating a treatment process by the device for inhibiting tumor promotion by using an electric field. In this process, the output voltage setting value is defined by u(t)=K1*t, where K1 is a positive proportional coefficient and t is time.

Due to the low absolute value of the output voltage setting value at this stage, the measured temperature value changes slowly. At the end of this process, the output voltage setting value may reach a certain set threshold, or the measured temperature value may reach a certain set threshold. Alternatively, in the early stage of initiating a treatment process by the device for inhibiting tumor proliferation by using an electric field, the curve of the output voltage setting value changing with time may also be a preset curve. Simpler control logic can be achieved by linear boosting.

After the linear boosting process is completed, if the output voltage setting value has become relatively high, the speed at which the output voltage setting value increases should be appropriately reduced. Therefore, the output voltage setting value can be updated according to the error value between the measured temperature value and the target temperature value.

For example, updating the output voltage setting value according to the error value between the measured temperature value and the target temperature value includes: updating the output voltage setting value according to the following formula:

$$u ( t ) = u ( t - 1 ) + K 2 * e ( t ) ,$$

where u(t−1) is the output voltage setting value for the current adjustment period, u(t) is the output voltage setting value for the next adjustment period, e(t) is the error value between the measured temperature value and the target temperature value in the current adjustment period, and K2 is a positive proportional coefficient.

The adjustment mechanisms provided by the above embodiments can be used in combination. For example, after the device for inhibiting tumor proliferation by using an electric field is initiated for treatment, linear boosting is firstly performed; then the output voltage setting value is updated according to the error value between the measured temperature value and the target temperature value (that is, dynamic boosting); when the measured temperature value falls within the target temperature range for the first time, the output voltage setting value is kept unchanged; if it is detected subsequently that the measured temperature value becomes lower than the lower limit of the target temperature range, the output voltage setting value is updated according to the error value between the measured temperature value and the target temperature value, and the differential value of the error value; if it is detected that the measured temperature value becomes greater than the upper limit of the target temperature range, the output voltage setting value is updated according to the error value between the measured temperature value and the target temperature value and the integral value of the error value. Once it is detected that the measured temperature value returns into the target temperature range, the output voltage setting value is kept unchanged.

In the above embodiments, the output voltage setting value is regulated based on the measured temperature value of the contact region between the skin and at least one of the electrode patches, the target temperature value, and the target temperature range, thus even if there is a significant fluctuation in the measured temperature value, the measured temperature value can be rapidly adjusted to return into the target temperature range, thereby keeping the output voltage setting value at a high level for a long time.

To achieve more precise adjustment of the output voltage setting value, in some embodiments, the method further includes acquiring an ambient temperature value of an environment where the human tissue is located. Accordingly, determining the output voltage setting value for the AC voltage source according to the measured temperature value, the target temperature value, and the target temperature range includes determining, according to the measured temperature value, the target temperature value, the target temperature range, and the ambient temperature value, the output voltage setting value for the AC voltage source. The ambient temperature value may affect the speed of heat dissipation of the electrode patches. Due to the introduction of the ambient temperature value as a parameter, the control of the output voltage setting value is more precise.

It should be noted that “an environment where the human tissue is located” here refers to an external environment where the human tissue is located, more accurately, an ambient temperature of the external environment of the skin in contact with the electrode patches.

In some embodiments, determining the output voltage setting value for the AC voltage source according to the measured temperature value, the target temperature value, the target temperature range, and the ambient temperature value includes:
after the measured temperature value falls within the target temperature range, if the measured temperature value becomes lower than the lower limit of the target temperature range, updating the output voltage setting value according to the error value between the measured temperature value and the target temperature value, the differential value of the error value, and a difference value between the measured temperature value and the ambient temperature value, where the integration start point for the integral value is located in an adjustment period in which the measured temperature value becomes greater than the upper limit of the target temperature range.

If the ambient temperature value is relatively greater than the target temperature value, the speed at which the output voltage setting value is raised should be appropriately reduced to prevent the measured temperature value from overshooting. If the ambient temperature value is relatively lower than the target temperature value, the speed at which the output voltage setting value is raised should be appropriately increased to prevent the measured temperature value from rising too slowly.

For example, updating the output voltage setting value according to the error value between the measured temperature value and the target temperature value, the differential value of the error value, and the difference value between the measured temperature value and the ambient temperature value includes: updating the output voltage setting value according to the following formula:

$$u ( t ) = u ( t - 1 ) + K {5}^{′} * e ( t ) + K {6}^{′} * de ( t ) / dt - Kt 3 * Δ T ,$$

where u(t−1) is the output voltage setting value for the current adjustment period, u(t) is the output voltage setting value for the next adjustment period, e(t) is the error value between the measured temperature value and the target temperature value in the current adjustment period, de(t)/dt is the differential value of the error value in the current adjustment period, ΔT is the difference value between the measured temperature value and the ambient temperature value in the current adjustment period, and K5′, K6′, and Kt3 are positive proportional coefficients.

Alternatively, the difference value between the measured temperature value and the ambient temperature value in the current adjustment process can also control the change trend of the measured temperature value in other ways such as dynamically influencing the values of the proportional coefficients K5′ and K6′.

In some embodiments, determining the output voltage setting value for the AC voltage source according to the measured temperature value, the target temperature value, the target temperature range, and the ambient temperature value includes:
after the measured temperature value falls within the target temperature range, if the measured temperature value becomes greater than the upper limit of the target temperature range, updating the output voltage setting value according to an error value between the measured temperature value and the target temperature value, an integral value of the error value, and a difference value between the measured temperature value and the ambient temperature value, where an integration start point for the integral value is located in an adjustment period in which the measured temperature value becomes greater than the upper limit of the target temperature range.

If the ambient temperature value is relatively greater than the target temperature value, the speed at which the output voltage setting value is reduced should be appropriately increased to prevent the measured temperature value from decreasing too slowly. If the ambient temperature value is relatively lower than the target temperature value, the speed at which the output voltage setting value is reduced should be appropriately reduced to prevent the measured temperature value from overshooting.

For example, updating the output voltage setting value according to the error value between the measured temperature value and the target temperature value, the integral value of the error value, and the difference value between the measured temperature value and the ambient temperature value includes: updating the output voltage setting value according to the following formula:

$$u ( t ) = u ( t - 1 ) + K 3 * e ( t ) + K {4}^{′} \int e ( t ) dt - Kt 2 * Δ T ,$$

where u(t−1) is the output voltage setting value for the current adjustment period, u(t) is the output voltage setting value for the next adjustment period, e(t) is the error value between the measured temperature value and the target temperature value in the current adjustment period, ∫e(t)dt is the integral value of the error value, ΔT is the difference value between the measured temperature value and the ambient temperature value in the current adjustment period, and K3′, K4′, and Kt2 are positive proportional coefficients.

Alternatively, the difference value between the measured temperature value and the ambient temperature value in the current adjustment process can also control the change trend of the measured temperature value in other ways such as dynamically influencing the values of the proportional coefficients K3′ and K4′.

In some embodiments, before determining the output voltage setting value for the AC voltage source according to the measured temperature value, the target temperature value, the target temperature range, and the ambient temperature value, the method further includes step 201 of linearly increasing the output voltage setting value for the AC voltage source over time.

Accordingly, determining the output voltage setting value between the pair of electrode patches according to the measured temperature value, the target temperature value, the target temperature range, and the ambient temperature value includes before the measured temperature value falls within the target temperature range for the first time, determining the output voltage setting value according to the error value between the measured temperature value and the target temperature value, and the difference value between the measured temperature value and the ambient temperature value.

In the linear heating process, the output voltage setting value is defined by u(t)=K1′*t, where K1′ is a positive proportional coefficient and t is time. Subsequently, the current ambient temperature value is further referred to for dynamic heating.

If the measured temperature value is relatively high, the speed at which the output voltage setting value increases should be appropriately reduced to avoid temperature overshoot. If the measured temperature value is relatively low, the speed at which the output voltage setting value increases should be appropriately increased to avoid a too slow increase in the output voltage.

For example, determining the output voltage setting value according to the error value between the measured temperature value and the target temperature value, and the difference value between the measured temperature value and the ambient temperature value includes: updating the output voltage setting value according to the following formula:

$$u ( t ) = u ( t - 1 ) + K {2}^{′} * e ( t ) - Kt 1 * Δ T ,$$

where u(t−1) is the output voltage setting value for the current adjustment period, u(t) is the output voltage setting value for the next adjustment period, e(t) is the error value between the measured temperature value and the target temperature value in the current adjustment period, ΔT is the difference value between the measured temperature value and the ambient temperature value in the current adjustment period, and K2′ and Kt1 are positive proportional coefficients.

Alternatively, the difference value of the measured temperature value and the ambient temperature value in the current adjustment process can also be used to control the change trend of the measured temperature value in other ways such as dynamically influencing the value of the proportional coefficient K2′.

The above control processes that introduce the ambient temperature value can also be used in combination. For example, after the device for inhibiting tumor proliferation by using an electric field is initiated for treatment, linear boosting is firstly performed; then the output voltage setting value is updated according to the error value between the measured temperature value and the target temperature value and the difference value between the measured temperature value and the ambient temperature value (that is, dynamic boosting). After the measured temperature value falls within the target temperature range for the first time, the output voltage setting value is kept unchanged. If it is detected subsequently that the measured temperature value is lower than the lower limit of the target temperature range, the output voltage setting value is updated according to the error value between the measured temperature value and the target temperature value, the differential value of the error value, and the difference value between the measured temperature value and the ambient temperature value. If it is detected that the measured temperature value is greater than the upper limit of the target temperature range, the output voltage setting value is updated according to the error value between the measured temperature value and the target temperature value, the integral value of the error value, and the difference value between the measured temperature value and the ambient temperature value. Once it is detected that the measured temperature value returns into the target temperature range, the output voltage setting value is kept unchanged.

There are other constraints on the control of the output voltage setting value for the device for inhibiting tumor proliferation by using an electric field. For example, the output voltage setting value cannot exceed the maximum allowable voltage value. For another example, after the measured temperature value falls within the target temperature range for the first time, the output voltage setting value cannot be lower than the minimum allowable voltage value.

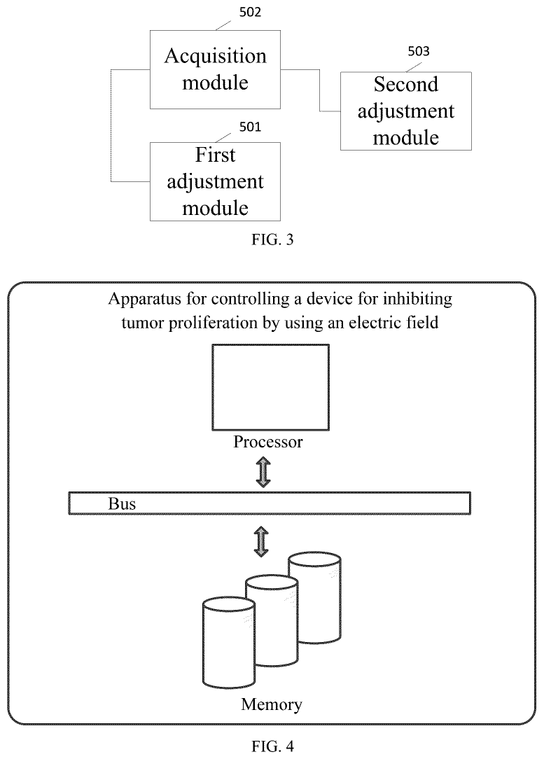

Based on the same technical concept, embodiments of the disclosure further provide an apparatus for controlling a device for inhibiting tumor proliferation by using an electric field, the apparatus being used to execute the control method provided by any of the above embodiments. FIG. 3 is a schematic structural diagram of the control apparatus provided in the embodiments of the disclosure.

As shown in FIG. 3, the control apparatus includes:
an acquisition module 502 configured to acquire a measured temperature value of a contact region between the skin 100 and at least one of the electrode patches;
a second adjustment module 503 configured to determine an output voltage setting value for the AC voltage source according to the measured temperature value, a target temperature value, and a target temperature range, where the target temperature value is within the target temperature range.

In some embodiments, the second adjustment module 503 is specifically configured to:
after the measured temperature value falls within the target temperature range, if the measured temperature value becomes lower than a lower limit of the target temperature range, update the output voltage setting value according to an error value between the measured temperature value and the target temperature value and a differential value of the error value.

In some embodiments, updating the output voltage setting value according to the error value between the measured temperature value and the target temperature value, and the differential value of the error value includes: updating the output voltage setting value according to the following formula:

$$u ( t ) = u ( t - 1 ) + K 5 * e ( t ) + K 6 * de ( t ) / dt ,$$

where u(t−1) is the output voltage setting value for the current adjustment period, u(t) is the output voltage setting value for the next adjustment period, e(t) is the error value between the measured temperature value and the target temperature value in the current adjustment period, de(t)/dt is the differential value of the error value in the current adjustment period, and K5 and K6 are positive proportional coefficients.

In some embodiments, the second adjustment module 503 is specifically configured to:
after the measured temperature value falls within the target temperature range, if the measured temperature value becomes greater than an upper limit of the target temperature range, update the output voltage setting value according to an error value between the measured temperature value and the target temperature value, and an integral value of the error value, where an integration start point for the integral value is located in an adjustment period in which the measured temperature value becomes greater than the upper limit of the target temperature range.

In some embodiments, updating the output voltage setting value according to the error value between the measured temperature value and the target temperature value and the integral value of the error value includes: updating the output voltage setting value according to the following formula:

$$u ( t ) = u ( t - 1 ) + K 3 * e ( t ) + K 4 \int e ( t ) dt ,$$

where u(t−1) is the output voltage setting value for the current adjustment period, u(t) is the output voltage setting value for the next adjustment period, e(t) is the error value between the measured temperature value and the target temperature value in the current adjustment period, ∫e(t)dt is the integral value of the error value, and K3 and K4 are positive proportional coefficients.

In some embodiments, the second adjustment module 503 is specifically configured to:
if the measured temperature value is within the target temperature range, maintain the output voltage setting value unchanged.

In some embodiments, the apparatus further includes: a first adjustment module 501 configured to, before determining the output voltage setting value for the AC voltage source according to the measured temperature value, the target temperature value, and the target temperature range, linearly increase the output voltage setting value for the AC voltage source over time; and the second adjustment module 503 is further specifically configured to, before the measured temperature value falls within the target temperature range for the first time, update the output voltage setting value according to the error value between the measured temperature value and the target temperature value.

In some embodiments, updating the output voltage setting value according to the error value between the measured temperature value and the target temperature value includes: updating the output voltage setting value according to the following formula:

$$u ( t ) = u ( t - 1 ) + K 2 * e ( t ) ,$$

where u(t−1) is the output voltage setting value for the current adjustment period, u(t) is the output voltage setting value for the next adjustment period, e(t) is the error value between the measured temperature value and the target temperature value in the current adjustment period, and K2 is a positive proportional coefficient.

In some embodiments, the acquisition module 502 is further configured to acquire an ambient temperature value of an environment where the human tissue is located; and the second adjustment module 503 is specifically configured to determine the output voltage setting value for the AC voltage source according to the measured temperature value, the target temperature value, the target temperature range, and the ambient temperature value.

In some embodiments, the second adjustment module 503 is specifically configured to:
after the measured temperature value falls within the target temperature range, if the measured temperature value becomes lower than the lower limit of the target temperature range, update the output voltage setting value according to the error value between the measured temperature value and the target temperature value, the differential value of the error value, and a difference value between the measured temperature value and the ambient temperature value, where the integration start point for the integral value is located in an adjustment period in which the measured temperature value becomes greater than the upper limit of the target temperature range.

In some embodiments, updating the output voltage setting value according to the error value between the measured temperature value and the target temperature value, the differential value of the error value, and the difference value between the measured temperature value and the ambient temperature value includes: updating the output voltage setting value according to the following formula:

$$u ( t ) = u ( t - 1 ) + K {5}^{′} * e ( t ) + K {6}^{′} * de ( t ) / dt - Kt 3 * Δ T ,$$

where u(t−1) is the output voltage setting value for the current adjustment period, u(t) is the output voltage setting value for the next adjustment period, e(t) is the error value between the measured temperature value and the target temperature value in the current adjustment period, de(t)/dt is the differential value of the error value in the current adjustment period, ΔT is the difference value between the measured temperature value and the ambient temperature value in the current adjustment period, and K5′, K6′, and Kt3 are positive proportional coefficients.

In some embodiments, the second adjustment module 503 is specifically configured to:
after the measured temperature value falls within the target temperature range, if the measured temperature value becomes greater than the upper limit of the target temperature range, update the output voltage setting value according to an error value between the measured temperature value and the target temperature value, an integral value of the error value, and a difference value between the measured temperature value and the ambient temperature value, where an integration start point for the integral value is located in an adjustment period in which the measured temperature value becomes greater than the upper limit of the target temperature range.

In some embodiments, updating the output voltage setting value according to the error value between the measured temperature value and the target temperature value, the integral value of the error value, and the difference value between the measured temperature value and the ambient temperature value includes: updating the output voltage setting value according to the following formula:

$$u ( t ) = u ( t - 1 ) + K 3 * e ( t ) + K {4}^{′} \int e ( t ) dt - Kt 2 * Δ T ,$$

where u(t−1) is the output voltage setting value for the current adjustment period, u(t) is the output voltage setting value for the next adjustment period, e(t) is the error value between the measured temperature value and the target temperature value in the current adjustment period, ∫e(t)dt is the integral value of the error value, ΔT is the difference value between the measured temperature value and the ambient temperature value in the current adjustment period, and K3′, K4′, and Kt2 are positive proportional coefficients.

In some embodiments, the apparatus further includes a first adjustment module 501 configured to, before determining the output voltage setting value for the AC voltage source according to the measured temperature value, the target temperature value, the target temperature range, and the ambient temperature value, linearly increase the output voltage setting value for the AC voltage source over time; and
the second adjustment module 503 is specifically configured to, before the measured temperature value falls within the target temperature range for the first time, determine the output voltage setting value according to the error value between the measured temperature value and the target temperature value, and the difference value between the measured temperature value and the ambient temperature value.

In some embodiments, determining the output voltage setting value according to the error value between the measured temperature value and the target temperature value, and the difference value between the measured temperature value and the ambient temperature value includes: updating the output voltage setting value according to the following formula:

$$u ( t ) = u ( t - 1 ) + K 2 * e ( t ) - Kt 1 * Δ T ,$$

where u(t−1) is the output voltage setting value for the current adjustment period, u(t) is the output voltage setting value for the next adjustment period, e(t) is the error value between the measured temperature value and the target temperature value in the current adjustment period, ΔT is the difference value between the measured temperature value and the ambient temperature value in the current adjustment period, and K2′ and Kt1 are positive proportional coefficients.

It should be noted that the control apparatus in the embodiments of the disclosure can implement the processes of the embodiments of the control method, and can achieve the same effects and functions, which is not described herein again.

FIG. 4 is an apparatus for controlling a device for inhibiting tumor proliferation by using an electric field according to an embodiment of the disclosure, the apparatus being used to execute the control method shown in FIG. 2. The control apparatus includes at least one processor and a memory communicatively connected to the at least one processor, the memory having instructions executable by the at least one processor stored therein, where the instructions, when executed by the at least one processor, cause the at least one processor to implement the aforesaid control method.

The embodiments of the disclosure further provide a device for inhibiting tumor proliferation by using an electric field. The device for inhibiting tumor proliferation by using an electric field includes a pair of electrode patches configured to be attached to a skin of a user, and an AC voltage source connected to the pair of electrode patches to apply an AC voltage to a human tissue of the user between the electrode patches, where the device for inhibiting tumor proliferation by using an electric field further includes the aforesaid control apparatus.

In some embodiments, the therapeutic instrument further includes a first temperature sensor and a second temperature sensor, where the first temperature sensor is configured to measure a measured temperature value of a contact region between the skin and at least one of the electrode patches of the device for inhibiting tumor proliferation by using an electric field, and the second temperature sensor is configured to measure an ambient temperature.

Various embodiments in the disclosure are described in a progressive manner, and the same and similar parts between the embodiments can be referred to each other. Each embodiment focuses on the differences from other embodiments. Especially for the embodiments of apparatuses and computer-readable storage media, their descriptions have been simplified due to their substantial similarity to method embodiments. For relevant information, please refer to the partial explanation of method embodiments.

The apparatus and method provided in the embodiments of the disclosure are in one-to-one correspondence, thus the apparatus also has beneficial technical effects similar to its corresponding method. Since the beneficial technical effects of the method have been explained in detail above, the beneficial technical effects of the apparatus will not be repeated here.

A person of ordinary skill in the art should understand that the embodiments of the disclosure may be provided as a method, a system, or a computer program product. Therefore, the present disclosure may be implemented as entirely hardware embodiments, entirely software embodiments, or embodiments with a combination of software and hardware. Moreover, the disclosure may be implemented as a computer program product embodied on one or more computer readable storage media (including but not limited to magnetic disk storages, CD-ROM, optical storages, etc.) containing computer readable program code.

Although the concepts and principles of the disclosure have been described with reference to several specific embodiments, it should be understood that the disclosure is not limited to the disclosed specific embodiments, and the division into various aspects does not mean that the features in these aspects cannot be combined for benefit. This division is only for the convenience of expression. The disclosure aims to cover various modifications and equivalent arrangements within the concepts and scope of the attached claims.

1. A method of controlling a device for inhibiting tumor proliferation by using an electric field, the device comprising a pair of electrode patches configured to be attached to a skin of a user and an alternating current (AC) voltage source connected to the pair of electrode patches to apply an AC voltage to a human tissue of the user between the electrode patches, wherein the method comprises:
acquiring a measured temperature value of a contact region between the skin and at least one of the electrode patches; and
determining an output voltage setting value for the AC voltage source according to the measured temperature value, a target temperature value, and a target temperature range, wherein the target temperature value is within the target temperature range.

2. The method according to claim 1, wherein the determining the output voltage setting value for the AC voltage source according to the measured temperature value, the target temperature value, and the target temperature range comprises:
after the measured temperature value falls within the target temperature range, if the measured temperature value becomes lower than a lower limit of the target temperature range, updating the output voltage setting value according to an error value between the measured temperature value and the target temperature value and a differential value of the error value.

3. The method according to claim 2, wherein the updating the output voltage setting value according to the error value between the measured temperature value and the target temperature value and the differential value of the error value comprises: updating the output voltage setting value as u(t)=u(t−1)+K5*e(t)+K6*de(t)/dt,
wherein u(t) is the output voltage setting value for a next adjustment period, u(t−1) is the output voltage setting value for a current adjustment period, e(t) is the error value between the measured temperature value and the target temperature value in the current adjustment period, de(t)/dt is the differential value of the error value in the current adjustment period, and K5 and K6 are positive proportional coefficients.

4. The method according to claim 1, wherein the determining the output voltage setting value for the AC voltage source according to the measured temperature value, the target temperature value, and the target temperature range comprises:
after the measured temperature value falls within the target temperature range, if the measured temperature value becomes greater than an upper limit of the target temperature range, updating the output voltage setting value according to an error value between the measured temperature value and the target temperature value and an integral value of the error value, wherein an integration start point for the integral value is located in an adjustment period in which the measured temperature value becomes greater than the upper limit of the target temperature range.

5. The method according to claim 4, wherein the updating the output voltage setting value according to the error value between the measured temperature value and the target temperature value and the integral value of the error value comprises: updating the output voltage setting value as u(t)=u(t−1)+K3*e(t)+K4∫e(t)dt,
wherein u(t) is the output voltage setting value for a next adjustment period, u(t−1) is the output voltage setting value for a current adjustment period, e(t) is the error value between the measured temperature value and the target temperature value in the current adjustment period, ∫e(t)dt is the integral value of the error value, and K3 and K4 are positive proportional coefficients.

6. The method according to claim 1, wherein the determining the output voltage setting value for the AC voltage source according to the measured temperature value, the target temperature value, and the target temperature range comprises:
if the measured temperature value is within the target temperature range, maintaining the output voltage setting value unchanged.

7. The method according to claim 1, wherein before determining the output voltage setting value for the AC voltage source according to the measured temperature value, the target temperature value, and the target temperature range, the method further comprises: linearly increasing the output voltage setting value for the AC voltage source over time, and
the determining the output voltage setting value for the AC voltage source according to the measured temperature value, the target temperature value, and the target temperature range comprises: before the measured temperature value falls within the target temperature range for the first time, updating the output voltage setting value according to an error value between the measured temperature value and the target temperature value.

8. The method according to claim 7, wherein the updating the output voltage setting value according to the error value between the measured temperature value and the target temperature value comprises: updating the output voltage setting value as u(t)=u(t−1)+K2*e(t), wherein u(t) is the output voltage setting value for a next adjustment period, u(t−1) is the output voltage setting value for a current adjustment period, e(t) is the error value between the measured temperature value and the target temperature value in the current adjustment period, and K2 is a positive proportional coefficient.

9. The method according to claim 1, further comprising: acquiring an ambient temperature value of an environment where the human tissue is located, and
determining the output voltage setting value for the AC voltage source according to the measured temperature value, the target temperature value, and the target temperature range comprises:
determining the output voltage setting value for the AC voltage source according to the measured temperature value, the target temperature value, the target temperature range, and the ambient temperature value.

10. The method according to claim 9, wherein the determining the output voltage setting value for the AC voltage source according to the measured temperature value, the target temperature value, the target temperature range, and the ambient temperature value comprises:
after the measured temperature value falls within the target temperature range, if the measured temperature value becomes lower than a lower limit of the target temperature range, updating the output voltage setting value according to an error value between the measured temperature value and the target temperature value, a differential value of the error value, and a difference value between the measured temperature value and the ambient temperature value.

11. The method according to claim 10, wherein the updating the output voltage setting value according to the error value between the measured temperature value and the target temperature value, the differential value of the error value, and the difference value between the measured temperature value and the ambient temperature value comprises: updating the output voltage setting value as u(t)=u(t−1)+K5′*e(t)+K6′*de(t)/dt−Kt3*ΔT,
wherein u(t) is the output voltage setting value for a next adjustment period, u(t−1) is the output voltage setting value for a current adjustment period, e(t) is the error value between the measured temperature value and the target temperature value in the current adjustment period, de(t)/dt is the differential value of the error value in the current adjustment period, ΔT is the difference value between the measured temperature value and the ambient temperature value in the current adjustment period, and K5′, K6′, and Kt3 are positive proportional coefficients.

12. The method according to claim 9, wherein the determining the output voltage setting value for the AC voltage source according to the measured temperature value, the target temperature value, the target temperature range, and the ambient temperature value comprises:
after the measured temperature value falls within the target temperature range, if the measured temperature value becomes greater than an upper limit of the target temperature range, updating the output voltage setting value according to an error value between the measured temperature value and the target temperature value, an integral value of the error value, and a difference value between the measured temperature value and the ambient temperature value, wherein an integration start point for the integral value is located in an adjustment period in which the measured temperature value becomes greater than the upper limit of the target temperature range.

13. The method according to claim 12, wherein the updating the output voltage setting value according to the error value between the measured temperature value and the target temperature value, the integral value of the error value, and the difference value between the measured temperature value and the ambient temperature value comprises: updating the output voltage setting value as u(t)=u(t−1)+K3′*e(t)+K4′∫e(t)dt−Kt2*ΔT,
wherein u(t) is the output voltage setting value for a next adjustment period, u(t−1) is the output voltage setting value for a current adjustment period, e(t) is the error value between the measured temperature value and the target temperature value in the current adjustment period, ∫e(t)dt is the integral value of the error value, ΔT is the difference value between the measured temperature value and the ambient temperature value in the current adjustment period, and K3′, K4′, and Kt2 are positive proportional coefficients.

14. The method according to claim 9, wherein before determining the output voltage setting value for the AC voltage source according to the measured temperature value, the target temperature value, the target temperature range, and the ambient temperature value, the method further comprises linearly increasing the output voltage setting value for the AC voltage source over time, and
determining the output voltage setting value for the AC voltage source according to the measured temperature value, the target temperature value, the target temperature range, and the ambient temperature value comprises:
before the measured temperature value falls within the target temperature range for the first time, determining the output voltage setting value according to an error value between the measured temperature value and the target temperature value and a difference value between the measured temperature value and the ambient temperature value.

15. The method according to claim 14, wherein the determining the output voltage setting value according to the error value between the measured temperature value and the target temperature value, and the difference value between the measured temperature value and the ambient temperature value comprises: updating the output voltage setting value as u(t)=u(t−1)+K2′*e(t)−Kt1*ΔT,
wherein u(t−1) is the output voltage setting value for a current adjustment period, u(t) is the output voltage setting value for a next adjustment period, e(t) is the error value between the measured temperature value and the target temperature value in the current adjustment period, ΔT is the difference value between the measured temperature value and the ambient temperature value in the current adjustment period, and K2′ and Kt1 are positive proportional coefficients.

16. An apparatus for controlling a device for inhibiting tumor proliferation by using an electric field, the device comprising a pair of electrode patches configured to be attached to a skin of a user and an alternating current (AC) voltage source connected to the pair of electrode patches to apply an AC voltage to a human tissue of the user between the electrode patches, wherein the apparatus comprises:
an acquisition module configured to acquire a measured temperature value of a contact region between the skin and at least one of the electrode patches; and
a second adjustment module configured to determine an output voltage setting value for the AC voltage source according to the measured temperature value, a target temperature value, and a target temperature range, wherein the target temperature value is within the target temperature range.

17. The apparatus according to claim 16, wherein the second adjustment module is configured to:
after the measured temperature value falls within the target temperature range, if the measured temperature value becomes lower than a lower limit of the target temperature range, update the output voltage setting value according to an error value between the measured temperature value and the target temperature value and a differential value of the error value.

18. The apparatus according to claim 17, wherein the updating the output voltage setting value according to the error value between the measured temperature value and the target temperature value, and the differential value of the error value comprises: updating the output voltage setting value as u(t)=u(t−1)+K5*e(t)+K6*de(t)/dt,
wherein u(t−1) is the output voltage setting value for a current adjustment period, u(t) is the output voltage setting value for a next adjustment period, e(t) is the error value between the measured temperature value and the target temperature value in the current adjustment period, de(t)/dt is the differential value of the error value in the current adjustment period, and K5 and K6 are positive proportional coefficients.

19. The apparatus according to claim 16, wherein the second adjustment module is configured to:
after the measured temperature value falls within the target temperature range, if the measured temperature value becomes greater than an upper limit of the target temperature range, updating the output voltage setting value according to an error value between the measured temperature value and the target temperature value, and an integral value of the error value, wherein an integration start point for the integral value is located in an adjustment period in which the measured temperature value becomes greater than the upper limit of the target temperature range.

20. The apparatus according to claim 19, wherein the updating the output voltage setting value according to the error value between the measured temperature value and the target temperature value and the integral value of the error value comprises: updating the output voltage setting value as u(t)=u(t−1)+K3*e(t)+K4∫e(t)dt,
wherein u(t−1) is the output voltage setting value for a current adjustment period, u(t) is the output voltage setting value for a next adjustment period, e(t) is the error value between the measured temperature value and the target temperature value in the current adjustment period, ∫e(t)dt is the integral value of the error value, and K3 and K4 are positive proportional coefficients.

21. The apparatus according to claim 16, wherein the second adjustment module is configured to:
if the measured temperature value is within the target temperature range, maintain the output voltage setting value unchanged.

22. The apparatus according to claim 16, further comprising: a first adjustment module configured to: before the determining the output voltage setting value for the AC voltage source according to the measured temperature value, the target temperature value, and the target temperature range, linearly increase the output voltage setting value for the AC voltage source over time, and
the second adjustment module is further configured to: before the measured temperature value falls within the target temperature range for the first time, update the output voltage setting value according to an error value between the measured temperature value and the target temperature value.

23. The apparatus according to claim 20, wherein the updating the output voltage setting value according to the error value between the measured temperature value and the target temperature value comprises: updating the output voltage setting value as u(t)=u(t−1)+K2*e(t),
wherein u(t−1) is the output voltage setting value for a current adjustment period, u(t) is the output voltage setting value for a next adjustment period, e(t) is the error value between the measured temperature value and the target temperature value in the current adjustment period, and K2 is a positive proportional coefficient.

24. The apparatus according to claim 16, wherein the acquisition module is further configured to acquire an ambient temperature value of an environment where the human tissue is located, and
the second adjustment module is configured to determine the output voltage setting value for the AC voltage source according to the measured temperature value, the target temperature value, the target temperature range, and the ambient temperature value.

25. The apparatus according to claim 24, wherein the second adjustment module is configured to:
after the measured temperature value falls within the target temperature range, if the measured temperature value becomes lower than a lower limit of the target temperature range, update the output voltage setting value according to an error value between the measured temperature value and the target temperature value, a differential value of the error value, and a difference value between the measured temperature value and the ambient temperature value.

26. The apparatus according to claim 25, wherein the updating the output voltage setting value according to the error value between the measured temperature value and the target temperature value, the differential value of the error value, and the difference value between the measured temperature value and the ambient temperature value comprises: updating the output voltage setting value as u(t)=u(t−1)+K5′*e(t)+K6′*de(t)/dt−Kt3*ΔT,
wherein u(t−1) is the output voltage setting value for a current adjustment period, u(t) is the output voltage setting value for a next adjustment period, e(t) is the error value between the measured temperature value and the target temperature value in the current adjustment period, de(t)/dt is the differential value of the error value in the current adjustment period, ΔT is the difference value between the measured temperature value and the ambient temperature value in the current adjustment period, and K5′, K6′, and Kt3 are positive proportional coefficients.

27. The apparatus according to claim 24, wherein the second adjustment module is configured to:
after the measured temperature value falls within the target temperature range, if the measured temperature value becomes greater than an upper limit of the target temperature range, update the output voltage setting value according to an error value between the measured temperature value and the target temperature value, an integral value of the error value, and a difference value between the measured temperature value and the ambient temperature value, wherein an integration start point for the integral value is located in an adjustment period in which the measured temperature value becomes greater than the upper limit of the target temperature range.

28. The apparatus according to claim 27, wherein the updating the output voltage setting value according to the error value between the measured temperature value and the target temperature value, the integral value of the error value, and the difference value between the measured temperature value and the ambient temperature value comprises: updating the output voltage setting value as the following formula:
$$u ( t ) = u ( t - 1 ) + K 3 * e ( t ) + K {4}^{′} \int e ( t ) dt - Kt 2 * Δ T ,$$
wherein u(t−1) is the output voltage setting value for a current adjustment period, u(t) is the output voltage setting value for a next adjustment period, e(t) is the error value between the measured temperature value and the target temperature value in the current adjustment period, ∫e(t)dt is the integral value of the error value, ΔT is the difference value between the measured temperature value and the ambient temperature value in the current adjustment period, and K3′, K4′, and Kt2 are positive proportional coefficients.

29. The apparatus according to claim 24, further comprising a first adjustment module configured to: before the determining the output voltage setting value for the AC voltage source according to the measured temperature value, the target temperature value, the target temperature range, and the ambient temperature value, linearly increase the output voltage setting value for the AC voltage source over time, and
the second adjustment module is configured to: before the measured temperature value falls within the target temperature range for the first time, determine the output voltage setting value according to an error value between the measured temperature value and the target temperature value and a difference value between the measured temperature value and the ambient temperature value.

30. The apparatus according to claim 29, wherein the determining the output voltage setting value according to the error value between the measured temperature value and the target temperature value, and the difference value between the measured temperature value and the ambient temperature value comprises: updating the output voltage setting value as u(t)=u(t−1)+K2′*e(t)−Kt1*ΔT,
wherein u(t−1) is the output voltage setting value for a current adjustment period, u(t) is the output voltage setting value for a next adjustment period, e(t) is the error value between the measured temperature value and the target temperature value in the current adjustment period, ΔT is the difference value between the measured temperature value and the ambient temperature value in the current adjustment period, and K2′ and Kt1 are positive proportional coefficients.

31. (canceled)

32. A device for inhibiting tumor proliferation by using an electric field, comprising a pair of electrode patches configured to be attached to a skin of a user and an alternating current (AC) voltage source connected to the pair of electrode patches to apply an AC voltage to a human tissue of the user between the electrode patches, wherein the device further comprises the apparatus according to claim 16.

33. The device for inhibiting tumor proliferation by using an electric field according to claim 32, further comprising a first temperature sensor and a second temperature sensor, wherein the first temperature sensor is configured to measure a temperature of a contact region between the skin and at least one of the electrode patches, and the second temperature sensor is configured to measure an ambient temperature.
